(19) United States
(12) Patent Application Publication
(10) Pub. No.: US 2025/0235281 A1
(43) Pub. Date: Jul. 24, 2025

(54) SURGICAL ASSISTANCE SYSTEM AND SURGICAL INSTRUMENT HOLDER THEREFOR

(71) Applicant: Ballast Medical Inc., Montreal (CA)

(72) Inventor: Michel GAGNER, Montreal (CA)

(21) Appl. No.: 19/027,957

(22) Filed: Jan. 17, 2025

Related U.S. Application Data
(60) Provisional application No. 63/622,114, filed on Jan. 18, 2024.

(51) Int. Cl. A61B 50/22; A61G 13/10

(52) U.S. Cl. CPC A61B 50/22; A61G 13/10

(57) ABSTRACT

A surgical assistance system for use in cooperation with a surgical table is provided. The surgical assistance system can include surgical instrument holders provided adjacent to each other to form an array configured for placement along a first longitudinal side of the surgical table, each surgical instrument holder comprising a peripheral wall having an upper end surface, the peripheral wall defining a surgical instrument receptacle having a surgical instrument receiving opening and being configured to extend downwardly once the surgical assistance system is engaged with the surgical table, the surgical instrument receptacle being configured to receive at least a portion of a surgical instrument therein.

### RELATED APPLICATION

This application claims priority to U.S. Provisional Patent Application Ser. No. 63/622,114, filed Jan. 18, 2024, and entitled SURGICAL ASSISTANCE SYSTEM AND SURGICAL INSTRUMENT HOLDER THEREFOR, the contents of which is incorporated in its entirety herein.

### TECHNICAL FIELD

The technical field relates to systems for assisting surgical procedures. More particularly, the technical field relates to a surgical assistance system that includes surgical instrument holders for receiving surgical instruments.

### BACKGROUND

A surgical procedure typically includes a series of steps involving the use of distinct surgical instruments. In the context of surgical procedures that do not rely on robotic systems, the series of steps are performed manually by a surgical care practitioner, such as a surgeon or a surgical assistant, who is generally limited to the manipulation of up to two surgical instruments at a given time. When each of a surgical care practitioner's hands are handling a surgical instrument, the use of an additional surgical instrument requires that the surgical care practitioner discharges a handheld surgical instrument before grasping a different surgical instrument.

These various surgical instruments are typically disposed on an instrument table adjacent to the surgical table for access by the surgical care practitioner. Such a placement of the surgical instruments necessitates that the surgical care practitioner shifts or extends their bodies away from the surgical table when seeking to exchange surgical instruments, which can lead to a temporary loss of focus and can introduce additional physical exertion during the surgical procedure.

The various surgical instruments are typically sterilized in accordance with sanitary surgical practices and are required to remain sterilized throughout the surgical procedure. The placement of the surgical instruments on the instrument table can lead to an increased risk of an unintentional contact with an unsterilized surface, such as when the surgical instrument is dropped. Such contact with an unsterilized surface may require a subsequent sterilization of the surgical instrument, thereby delaying the surgical procedure and increasing the costs associated therewith.

Accordingly, there remain a number of challenges with respect to accessing surgical instruments during surgical procedures, and in particular, challenges related to objects or physical structures enabling a surgical care practitioner to access surgical instruments during a surgical procedure.

### SUMMARY

According to one aspect, there is provided a surgical assistance system for use in cooperation with a surgical table, the surgical assistance system comprising:
surgical instrument holders provided adjacent to each other to form an array configured for placement along a first longitudinal side of the surgical table, each surgical instrument holder comprising:
a peripheral wall having an upper end surface, the peripheral wall defining a surgical instrument receptacle having a surgical instrument receiving opening and being configured to extend downwardly once the surgical assistance system is engaged with the surgical table, the surgical instrument receptacle being configured to receive at least a portion of a surgical instrument therein.

In some implementations, the surgical assistance system further comprises a brace for hanging the array of surgical instrument holders, the brace being configured to extend along at least a portion of the first longitudinal side of the surgical table and including first and second end portions engageable with the surgical table.

In some implementations, each one of the surgical instrument holders includes a brace engaging feature engageable with the brace.

In some implementations, the brace is flexible.

In some implementations, the brace is rigid.

In some implementations, the surgical assistance system further comprises first and second siderail engaging members configured to secure the first and second end portions of the brace, respectively, to a siderail of the surgical table.

In some implementations, the surgical assistance system further comprises first and second drape engaging members configured to secure the first and second end portions of the brace, respectively, to a surgical drape.

In some implementations, one of the surgical instrument holders is removably engageable with an adjacent one of the surgical instrument holders of the array of surgical instrument holders.

In some implementations, the one of the surgical instrument holders comprises an attachment component coupled to the peripheral wall, the attachment component enabling the removable engagement of the one of the surgical instrument holders with the adjacent one of the surgical instrument holders.

In some implementations, the attachment component is one or more of a clip, a hook, and a magnetic connector.

In some implementations, a foremost one of the surgical instrument holders and an endmost one of the surgical instrument holders of the array of surgical instrument holders are removably engageable with the surgical table.

In some implementations, the surgical assistance system further comprises first and second engaging members configured to removably secure the foremost one of the surgical instrument holders of the array of surgical instrument holders and the endmost one of the surgical instrument holders of the array of surgical instrument holders, respectively, to the surgical table.

In some implementations, the first engaging member is a siderail engaging member configured to removably secure the foremost one of the surgical instrument holders to a siderail of the surgical table.

In some implementations, the first engaging member is a drape engaging member configured to removably secure the foremost one of the surgical instrument holders to a surgical drape of the surgical table.

In some implementations, each of the first and second engaging members is removably engageable with the attachment component of the foremost and endmost ones of the surgical instrument holders, respectively.

In some implementations, the array of surgical instrument holders is a first array of surgical instrument holders, the surgical assistance system further comprising a second array of surgical instrument holders configured for placement along a second longitudinal side of the surgical table opposite the first longitudinal side of the surgical table.

In some implementations, the surgical assistance system further comprises first and second support bands configured to extend across at least a portion of a width of the surgical table between the first and second longitudinal sides of the surgical table, the first and second support bands being longitudinally spaced apart along a longitudinal axis of the surgical table once the surgical assistance system is engaged with the surgical table, wherein each of the first and second arrays of surgical instrument holders is engageable with the first and second support bands.

In some implementations, the first and second support bands are configured to extend over a portion of a body of a patient positioned on the surgical table, the portion of the body supporting the first and second support bands.

In some implementations, the surgical instrument receptacle of a respective one of the surgical instrument holders extends along a longitudinal axis of the respective one of the surgical instrument holders.

In some implementations, the surgical instrument receptacle of a respective one of the surgical instrument holders is an open-top surgical instrument receptacle and the surgical instrument receiving opening is located at an upper end of the respective one of the surgical instrument holders.

In some implementations, the surgical instrument receptacle of a respective one of the surgical instrument holders is a closed-top surgical instrument receptacle and the surgical instrument receiving opening is defined in a sidewall of the peripheral wall of the respective one of the surgical instrument holders.

In some implementations, the surgical instrument receiving opening of a respective one of the surgical instrument holders has an opening width larger than a transverse instrument width of the surgical instrument to receive the surgical instrument free of interference within the surgical instrument receptacle of the respective one of the surgical instrument holders.

In some implementations, the opening width of the surgical instrument receiving opening is smaller than a widest transverse instrument width of at least a portion of the surgical instrument to enable the at least a portion of the surgical instrument to abut the upper end surface of the respective one of the surgical instrument holders while enabling insertion of a remainder of the surgical instrument into the surgical instrument receptacle of the respective one of the surgical instrument holders.

In some implementations, the surgical instrument receiving opening and the surgical instrument receptacle of at least one of the surgical instrument holders is shaped and dimensioned to receive one or more of a laparoscopic irrigation cannula, ultrasonic laparoscopic forceps, a surgical needle driver, a laparoscopic bowel grasper, a monopolar hook, and surgical scissors.

In some implementations, at least one of the surgical instrument holders includes a retainer member provided in a lower portion thereof to retain the surgical instrument within the respective surgical instrument receptacle.

In some implementations, adjacent surgical instrument holders of the array of surgical instrument holders extend downwardly at corresponding angles with respect to a vertical axis once the surgical assistance system is engaged with the surgical table to facilitate an insertion of the surgical instrument into the respective surgical instrument receptacle.

In some implementations, adjacent surgical instrument holders of the array of surgical instrument holders extend downwardly at progressively increasing angles with respect to the vertical axis once the surgical assistance system is engaged with the surgical table to facilitate an insertion of the surgical instrument into the respective surgical instrument receptacle.

In some implementations, at least one of the surgical instrument holders comprises an interior lining covering an interior surface of the peripheral wall.

In some implementations, the interior lining is made of a low friction material configured to reduce wear to the surgical instrument when the surgical instrument is received into the surgical instrument receptacle.

In some implementations, the interior lining is made of an electrically non-conductive material.

In some implementations, at least one of the surgical instrument holders is made of an electrically non-conductive material.

In some implementations, at least one of the surgical instrument holders extends downwardly at an angle with respect to a vertical axis once the surgical assistance system is engaged with the surgical table to facilitate an insertion of the surgical instrument into a corresponding surgical instrument receptacle.

In some implementations, the at least one of the surgical instrument holders extends downwardly at an angle of at least 5° with respect to the vertical axis once the surgical assistance system is engaged with the surgical table.

In some implementations, the at least one of the surgical instrument holders extends downwardly at an angle of at least 10° with respect to the vertical axis once the surgical assistance system is engaged with the surgical table.

In some implementations, the at least one of the surgical instrument holders extends downwardly at an angle of at least 20° with respect to the vertical axis once the surgical assistance system is engaged with the surgical table.

In some implementations, at least one of the surgical instrument holders is made of one or more of aluminum, stainless steel, and a sterilizable medical-grade polymer.

In some implementations, the at least one of the surgical instrument holders is made of one or more of polyether ether ketone, polyphenylsulfone, polyoxymethylene, and polypropylene.

In some implementations, each one of the surgical instrument holders is sterilizable.

In some implementations, the peripheral wall of at least one of the surgical instrument holders includes modular parts configured to be assembled to form a corresponding one of the surgical instrument holders.

In some implementations, at least one of the surgical instrument holders has a cylindrical shape.

In some implementations, a pair of surgical instrument holders of the array of surgical instrument holders are horizontally spaced apart and together define a horizontal spacing providing access to the surgical table once the surgical assistance system is engaged with the surgical table.

In some implementations, the horizontal spacing is at least 45 cm.

In some implementations, the horizontal spacing is at least 60 cm.

In some implementations, the surgical assistance system is a modular assembly configured to include a variable number of surgical instrument holders of the surgical assistance system.

In some implementations, the surgical instrument holders extend downwardly from the surgical table in a substantially parallel direction relative to each other once the surgical assistance system is engaged with the surgical table.

According to another aspect, there is provided a surgical instrument holder for use in cooperation with a surgical table, the surgical instrument holder comprising:
a peripheral wall defining a surgical instrument receptacle having a surgical instrument receiving opening, the surgical instrument receptacle being sized to receive at least a portion of a surgical instrument therein; and
an attachment component coupled to the peripheral wall, the attachment component being removably engageable with a corresponding attachment component of an adjacent surgical instrument holder to form an array of surgical instrument holders, the array of surgical instrument holders being configured for placement along a first longitudinal side of the surgical table.

In some implementations, the surgical instrument receptacle is configured to extend downwardly once the surgical instrument holder is engaged with the surgical table.

In some implementations, the attachment component is removably engageable with an engaging member, the engaging member being configured to engage with the surgical table via one or more of a surgical drape and a rail of the surgical table.

In some implementations, the surgical instrument receptacle is an open-top surgical instrument receptacle and the surgical instrument receiving opening is located at an upper end of the surgical instrument holder.

In some implementations, the surgical instrument receptacle is a closed-top surgical instrument receptacle and the surgical instrument receiving opening is located in a sidewall of the peripheral wall.

In some implementations, the surgical instrument receiving opening has an opening width larger than a transverse width of the surgical instrument to receive the surgical instrument free of interference within the surgical instrument receptacle.

In some implementations, the opening width of the surgical instrument receiving opening is smaller than a widest transverse width of at least a portion of the surgical instrument to enable the at least a portion of the surgical instrument to abut an upper end surface of the surgical instrument holder while enabling insertion of a remainder of the surgical instrument into the surgical instrument receptacle.

In some implementations, the surgical instrument receiving opening and the surgical instrument receptacle are shaped and dimensioned to receive one or more of a laparoscopic irrigation cannula, ultrasonic laparoscopic forceps, a surgical needle driver, a laparoscopic bowel grasper, a monopolar hook, and surgical scissors.

According to another aspect, there is provided a surgical assistance system for use in cooperation with a surgical table, the surgical assistance system comprising:
a surgical instrument holder comprising a peripheral wall defining a surgical instrument receptacle, the surgical instrument receptacle being configured to receive at least a portion of a surgical instrument therein; and
a support band having first and second band ends, the support band being configured to extend across at least a portion of a width of the surgical table between opposite first and second longitudinal sides of the surgical table;
wherein the surgical instrument holder is configured to be engaged with the first band end of the support band so that the surgical instrument holder is provided on the first longitudinal side of the surgical table once the support band is mounted to the surgical table.

In some implementations, the support band is configured to extend over a portion of a body of a patient positioned on the surgical table, the portion of the body supporting the support bands.

In some implementations, the surgical instrument receptacle is configured to extend downwardly once the surgical instrument holder is engaged with the support band.

In some implementations, the first band end of the support band is configured to be engaged with an upper portion of the surgical instrument holder to hang the surgical instrument holder in an upright disposition.

In some implementations, an upper portion of the surgical instrument holder is engageable with the support band to suspend the surgical instrument holder at an angle with respect to a vertical axis once the support band is mounted to the surgical table to facilitate an insertion of the surgical instrument into the surgical instrument receptacle.

In some implementations, the surgical instrument holder is a first surgical instrument holder, the surgical assistance system further comprising a second surgical instrument holder configured to be engaged with the second band end of the support band so as to be suspended from the support band on the second longitudinal side of the surgical table once the support band is mounted to the surgical table.

In some implementations, the support band is a first support band, the surgical assistance system further comprising a second support band being horizontally spaced apart from the first support band along a longitudinal axis of the surgical table once the first and second support bands are mounted to the surgical table.

In some implementations, the surgical assistance system further comprises third and fourth surgical instrument holders, the third and fourth surgical instrument holders being configured to be engaged with the first and second band ends of the second support band, respectively, so as to be suspended from the second support band on the first and second longitudinal sides of the surgical table, respectively, once the second support band is mounted to the surgical table.

In some implementations, the surgical assistance system further comprises additional surgical instrument holders provided between the first and third surgical instrument holders to form a first array of surgical instrument holders and/or between the second and fourth surgical instrument holders to form a second array of surgical instrument holders.

In some implementations, one or more of the surgical instrument holders of the additional surgical instrument holders includes one or more features as defined above.

In some implementations, two adjacent ones of the surgical instrument holders are removably engageable with each other.

In some implementations, a foremost surgical instrument holder and an endmost surgical instrument holder of the first array of surgical instrument holders are removably engageable with the first surgical instrument holder and the third surgical instrument holder, respectively.

In some implementations, a foremost surgical instrument holder and an endmost surgical instrument holder of the second array of surgical instrument holders are removably engageable with the second surgical instrument holder and the fourth surgical instrument holder, respectively.

According to another aspect, there is provided a surgical assistance system for use in cooperation with a surgical table having a top surface for receiving a patient thereon, the surgical assistance system comprising:
a surgical instrument holder comprising a peripheral wall defining a surgical instrument receptacle having a surgical instrument receiving opening, the surgical instrument receptacle being configured to receive at least a portion of a surgical instrument therein; and
a brace configured to extend across at least a portion of a width of the surgical table between longitudinal sides of the surgical table and to be vertically spaced-apart from the top surface of the surgical table to define a brace gap therebetween, the brace being engageable with the surgical instrument holder.

In some implementations, the brace gap is selected such that once the patient is positioned on the top surface of the surgical table, the brace remains spaced apart from the patient, in an upward direction.

In some implementations, the surgical instrument receptacle of the surgical instrument holder extends substantially parallelly relative to the top surface of the surgical table when the brace extends across the at least a portion of the width of the surgical table.

In some implementations, the surgical instrument receptacle of the surgical instrument holder extends at an angle relative to the top surface of the surgical table when the brace extends across the at least a portion of the width of the surgical table.

In some implementations, the brace is rigid or semi-rigid.

In some implementations, the brace is configurable between a collapsed configuration and a deployed configuration, the brace being engageable with the surgical instrument holder when in the deployed configuration.

In some implementations, when the brace is in the deployed configuration, the brace is a freestanding brace.

In some implementations, the brace includes first and second end portions engageable with the surgical table.

In some implementations, surgical assistance system further comprises first and second siderail engaging members configured to secure the first and second end portions of the brace, respectively, to a siderail of the surgical table.

In some implementations, the surgical assistance system further comprises first and second drape engaging members configured to secure the first and second end portions of the brace, respectively, to a surgical drape of the surgical table.

### BRIEF DESCRIPTION OF THE DRAWINGS

The attached figures illustrate various features, aspects and implementations of the technology described herein.

FIG. 1 is a top perspective view of a surgical instrument holder of a surgical assistance system, in accordance with an implementation, shown adjacent to a surgical instrument.

FIG. 2 is a top perspective view of the surgical instrument holder of FIG. 1, shown with the surgical instrument received therein.

FIG. 3 is a top perspective view of a surgical instrument holder of a surgical assistance system, in accordance with another implementation.

FIG. 4 is a bottom perspective view of a surgical instrument holder, in accordance with another implementation.

FIG. 5 is a top perspective view of a brace of a surgical assistance system, in accordance with an implementation, shown supporting the surgical instrument holder of FIG. 1.

FIG. 6 is a top perspective view of the surgical assistance system of FIG. 5, including additional surgical instrument holders and siderail engaging members, in accordance with an implementation.

FIG. 7 is a top perspective view of the surgical assistance system of FIG. 5, including additional surgical instrument holders and drape engaging members, in accordance with an implementation.

FIG. 8 is a perspective view of a surgical assistance system, in accordance with an implementation, shown engaged with a surgical table via a surgical drape, the surgical table supporting a patient.

FIG. 9 is a perspective view of a brace and a siderail engaging member of the surgical assistance system, in accordance with an implementation, shown engaged with a surgical table via a surgical rail.

FIG. 10 is a perspective view of a surgical assistance system, in accordance with another implementation, shown engaged with a surgical table via support bands of the surgical assistance system, the surgical table supporting a patient.

FIG. 11 is a top perspective view of a surgical assistance system, in accordance with another implementation, shown engaged with a surgical table via a surgical rail.

FIG. 12 is a top perspective view of a surgical assistance system, in accordance with another implementation.

FIG. 13 is a top perspective view of a surgical assistance system including two subsets of surgical instrument holders, in accordance with another implementation.

FIG. 14 is a perspective view of a surgical assistance system including two subsets of surgical instrument holders, in accordance with another implementation, shown engaged with a surgical table via a surgical rail, the surgical table supporting a patient.

FIG. 15 is a perspective view of a surgical assistance system including two subsets of surgical instrument holders, in accordance with another implementation, shown engaged with a surgical table via a surgical rail, the surgical table supporting a patient and a surgical care practitioner being positioned along a longitudinal side of the surgical table.

### DETAILED DESCRIPTION

Techniques described herein relate to systems, devices and methods for facilitating access to one or more surgical instruments during a surgical procedure.

In some implementations, it can be desirable to facilitate the exchange of surgical instruments during a surgical procedure by providing access to each of the necessary surgical instruments to a surgical care practitioner while the surgical care practitioner remains relatively stationary in an operative position (i.e., a position held by the surgical care practitioner during the surgical procedure) without requiring the surgical care practitioner to significantly shift their body or extend their arms away from the surgical table. The improved access to the surgical instruments can be achieved by providing a surgical assistance system including one or more surgical instrument holders along a longitudinal side of the surgical table within arms reach of the surgical care practitioner. In particular, the surgical assistance system can engage the surgical table in an area proximate the operative position of the surgical care practitioner to provide access to one or more surgical instruments without requiring them to significantly shift their body away from the operative position.

During a surgical procedure, a surgical table typically defines a sterilized environment including surfaces which have undergone a sterilization process in preparation for the surgical procedure and is therefore free from bacteria or other living organisms. The environment surrounding the surgical table can be considered as an unsterilized environment including surfaces which may not have undergone a sterilization process in preparation for the surgical procedure and therefore may not be free from bacteria or other living organisms. For instance, the surgical table can include a siderail forming part of the unsterilized environment and configured to mountingly receive one or more surgical table accessories. Examples of surgical table accessories configured to be mounted to the siderail of the surgical table include arm supports, body restraints, head rests, leg supports, accessory carts, displays, lighting fixtures, liver retractors, abdominal wall retractors, camera holders, and robotic arms, among others. The surgical table can further include a surgical drape forming part of the sterilized environment and configured to maintain a sterile field at the surgical site. In some implementations, the surgical assistance system as described herein can be configured to engage a surgical table at a location within the sterilized environment or the unsterilized environment.

It will be appreciated that positional descriptions such as “above”, “below”, “left”, “right”, “inwardly”, “outwardly” and the like should, unless otherwise indicated, be taken in the context of the figures, and should not be considered limiting. The term “outwardly” is intended to refer to a feature that extends toward an exterior side of a reference axis. The term “inwardly” is intended to refer to a feature that extends toward an interior side of a reference axis. It should also be understood that elongated objects described herein are considered to have an implicit “longitudinal axis” and “lateral axis”. The expression “longitudinal axis” is intended to refer to an axis extending along the length of the object, and the expression “lateral axis” is intended to refer to an axis extending perpendicularly to the longitudinal axis, along the width of the object. When referring to a longitudinal direction, it is intended to refer to a direction that extends substantially parallel to the longitudinal axis of the object, encompassing as well as directions that deviate slightly from the longitudinal axis. When referring to a lateral direction, it is intended to refer to a direction that extends substantially parallel to the lateral axis of the object, encompassing as well as directions that deviate slightly from the lateral axis.

Various implementations and features of the surgical instrument holder will now be described in greater detail in the following paragraphs. General description of the surgical assistance system

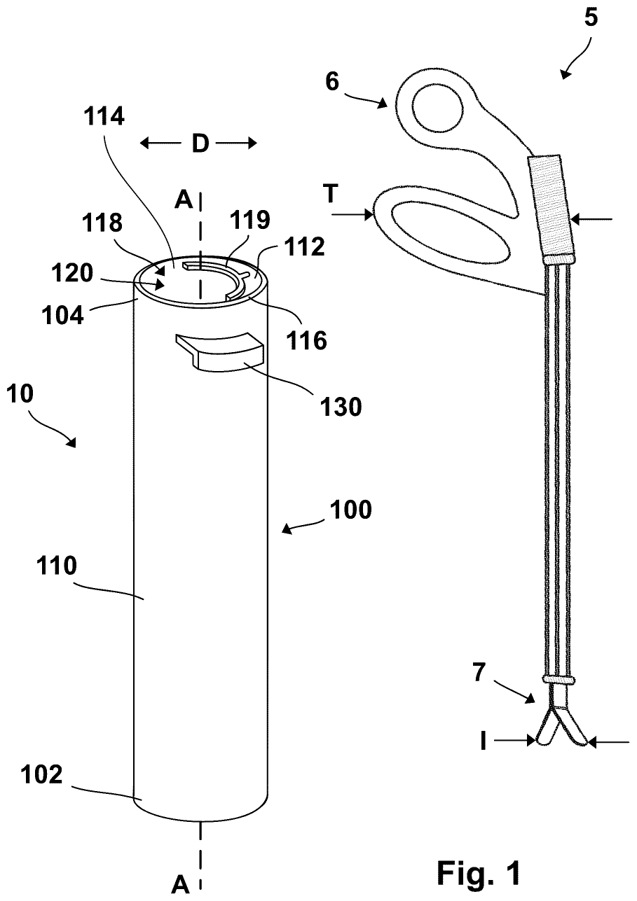

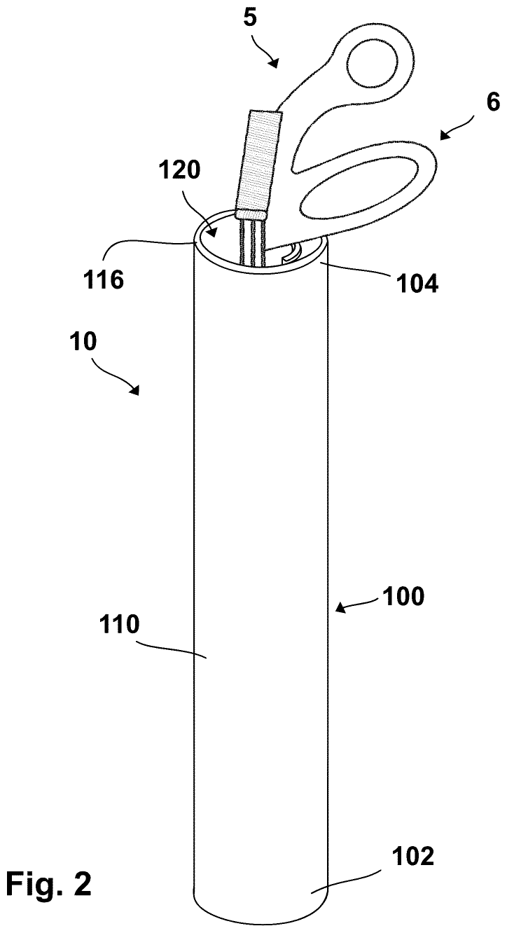

With reference to FIGS. 1 and 2, a surgical assistance system 10 for use in cooperation with a surgical table is shown. The surgical assistance system 10 includes a surgical instrument holder 100 configured to retain a surgical instrument 5 that can be used in the context of a surgical procedure. Examples of surgical instruments which the surgical instrument holder 100 can be configured to receive include surgical scissors, forceps, scalpels, clamps, needles, needle drivers, sutures, retractors, distractors, trocars, dilators, specula, bowel graspers, surgical staplers, irrigation and injection needles, endoscopes, monopolar hooks. laparoscopic irrigation cannulas, ultrasonic laparoscopic forceps, and monopolar hooks, among others.

The surgical instrument holder 100 includes a peripheral wall 110 having an upper end surface 116. In the implementation shown, the peripheral wall 110 defines a surgical instrument receptacle 120 that is substantially tubular, the surgical instrument receptacle 120 having a surgical instrument receiving opening 118 at an upper end 104 of the surgical instrument holder 100. The surgical instrument receptacle 120 shown in FIGS. 1 and 2 can thus be referred to as an open-top surgical instrument receptacle 120. The surgical instrument receptacle 120 is configured to receive at least a portion of the surgical instrument 5 therein via the surgical instrument receiving opening 118. In some implementations, the peripheral wall 110 can include a bottom wall (not shown) at a lower end 102 of the surgical instrument holder 100 to further define the surgical instrument receptacle 120. The bottom wall can be solid throughout, or can take the form of a grid for instance. In the implementation shown in FIGS. 1 and 2, the surgical instrument receptacle 120 thus extends between the lower and upper ends 102, 104 of the surgical instrument holder 100 along a longitudinal axis A thereof.

In the implementation shown in FIGS. 1 and 2, the surgical instrument receiving opening 118 is thus located at the upper end 104 of the surgical instrument holder 100 for providing access to the surgical instrument receptacle 120. The shape and dimension of the surgical instrument receiving opening 118 and the surgical instrument receptacle 120 can depend on the surgical instrument 5, or a portion thereof, that is intended to be received within the surgical instrument receptacle 120. For instance, in some implementations, the surgical instrument receiving opening 118 can have an opening width D which is larger than a widest transverse instrument width T of the surgical instrument 5 to receive the surgical instrument 5 free of interference within the surgical instrument receptacle 120. In the context of the present description, the expression “free of interference” is intended to mean that the opening width D is sufficiently large such that the entirety of the surgical instrument 5 can fit within the surgical instrument receptacle 120. In such implementations, given that no portion of the surgical instrument 5 has a transverse instrument width T that is larger than the opening width D, the peripheral wall 110 of the surgical instrument receptacle 120 can include a bottom wall to retain the surgical instrument 5 within the surgical instrument receptacle 120. It will be appreciated that, in some implementations, the opening width D of the surgical instrument receiving opening 118 can correspond to an internal width of the remainder of the surgical instrument receptacle 120 while, in other implementations, the opening width D of the surgical instrument receiving opening 118 can be greater or smaller than the internal width of the remainder of the surgical instrument receptacle 120. In some implementations, each of the opening width D of the surgical instrument receiving opening 118 and a width of the surgical instrument receptacle 120 can be between about 0.5 cm and about 10 mm. In some implementations, each of the opening width D of the surgical instrument receiving opening 118 and the width of the surgical instrument receptacle 120 can be between about 2 cm and about 8 cm. In some implementations, each of the opening width D of the surgical instrument receiving opening 118 and the width of the surgical instrument receptacle 120 can be between about 4 cm and about 6 cm.

In some implementations, the opening width D of the surgical instrument receiving opening 118 can be larger or smaller than the widest transverse instrument width T of the surgical instrument 5, and the surgical instrument receptacle 120 can have a receptacle height that is smaller than an instrument height to enable a graspable portion 6 of the surgical instrument 5 to remain easily accessible to the surgical care practitioner while a remainder of the surgical instrument 5 is inserted into the surgical instrument receptacle 120.

In some implementations, the opening width D of the surgical instrument receiving opening 118 can be smaller than a widest transverse instrument width T of the surgical instrument 5 to enable an upper portion of the surgical instrument 5 to abut the upper end surface 116 of the surgical instrument holder 110 when the surgical instrument 5 is placed upright in the surgical instrument receptacle 120. The resulting interference between the upper end surface 116 and the upper portion of the surgical instrument 5 can prevent an insertion of the graspable portion 6 of the surgical instrument 5 into the surgical instrument receptacle 120 (as shown in FIG. 2) such that the graspable portion 6 remains easily accessible to the surgical care practitioner while a remainder of the surgical instrument 5 is inserted into the surgical instrument receptacle 120.

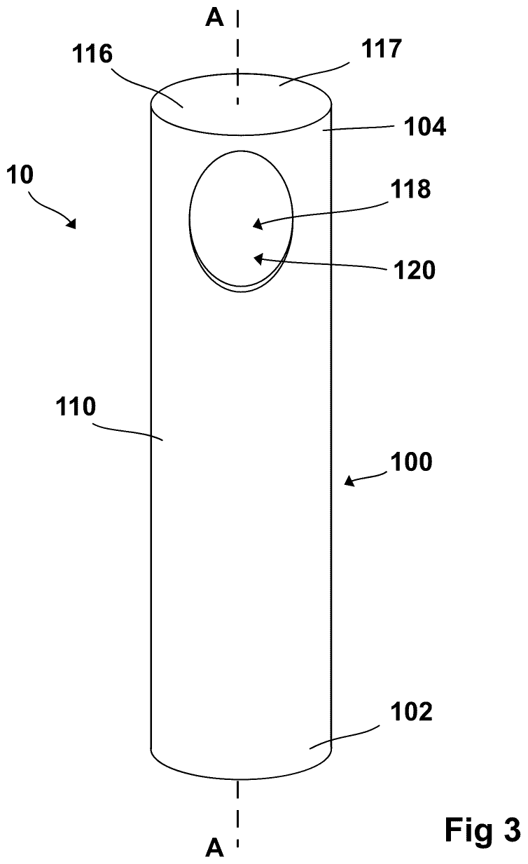

Referring to FIG. 3, another implementation of a surgical instrument holder 100 is shown. In this implementation, the peripheral wall 110 includes a top wall 117 that contributes to define a surgical instrument receptacle 120, with the bottom wall as described above being optionally present or not. The surgical instrument receptacle 120 shown in FIG. 3 can thus be referred to as a closed-top surgical instrument receptacle 120. The top wall 117 has an upper end surface 116 that spans across an entire width of the surgical instrument holder 100 and thus defines an upper end 104 that is closed. In this implementation, a sidewall of the peripheral wall 110 defines the surgical instrument receiving opening 118 proximate to the upper end surface 116 of the surgical instrument holder 100 to provide access to the surgical instrument receptacle 120.

It will be appreciated that while the implementations of the surgical instrument holder 100 shown in FIGS. 1 to 3 are exemplified as having a substantially cylindrical peripheral wall 110 with a circular cross-section along the longitudinal axis A of the surgical instrument holder 100, the surgical instrument holder 100 can have any other suitable shape. Thus, in other implementations, the transverse cross-section of the surgical instrument holder 100 can be shaped as elliptic, triangular, rectangular, pentagonal, hexagonal, heptagonal, or octagonal, for instance. In such implementations, the opening width D of the surgical instrument receiving opening 118 and the width of the surgical instrument receptacle 120 can be interpreted as corresponding to a width defined between two furthest points along the outer periphery of the sidewall of the peripheral wall 110.

The surgical instrument holder 100 can be configured to receive, within the surgical instrument receptacle 120, at least a portion of a surgical instrument 5 that is intended for contact with sterile body tissues or fluids. In some implementations, the peripheral wall 110 can therefore be made of a sterilizable material to prevent contamination during a given surgical procedure. The peripheral wall 110 can be made of a material that is generally suitable for sterilization by one or more sterilization processes including, steam sterilization, flash sterilization, ethylene oxide sterilization, hydrogen peroxide gas plasma sterilization, peracetic acid sterilization, glass bead sterilization, and the like. In some implementations, the peripheral wall 110 can be made of a metal, a polymer, or combinations thereof. Examples of suitable metals that can be used for the peripheral wall 110 include aluminum, stainless steel, titanium and nitinol. Examples of suitable polymers that can be used for the peripheral wall 110 can include polyether ether ketone, polyphenylsulfone, polyoxymethylene, and polypropylene, among others.

In some implementations, the surgical instrument holder 100 can be configured to receive at least a portion of a surgical instrument 5 using an electrical current, such as an electrosurgery instrument. Accordingly, the peripheral wall 110 can be made of one or more of the materials presented above having electrically non-conductive properties to prevent an unintentional electrocution of the surgical care practitioner coming into contact with the surgical instrument holder 100.

In some implementations, the surgical instrument holder 100 can be configured to receive a surgical instrument 5 having an operative feature 7 (shown in FIG. 1) at a distal portion thereof. In such implementations, the operative feature 7 can be the first portion of the surgical instrument 5 to be inserted into the surgical instrument receptacle 120 when the surgical instrument 5 is received within the respective surgical instrument holder 100. Accordingly, the operative feature 7 of the surgical instrument 5 can potentially contact and/or scrape against an interior surface 112 of the peripheral wall 110 which can abrade the operative feature 7 of the surgical instrument 5. In some implementations, the surgical instrument holder 100 can include an interior lining 114 covering at least a portion of the interior surface 112 of the peripheral wall 110. The interior lining 114 can be made of a low friction material configured to reduce wear to the surgical instrument 5 when the surgical instrument 5 is received into the surgical instrument receptacle 120 and, particularly, to the operative feature 7 of the surgical instrument 5. In some implementations, the interior lining 114 can be made of an electrically non-conductive material to prevent an unintentional electrocution of the surgical care practitioner. Examples of suitable materials include Teflon™, polycarbonate, silicone, polytetrafluoroethylene (PTFE), titanium and ceramics, among others.

### Retainer Member

Referring again to FIG. 1, in some implementations, the surgical instrument holder 100 can include a retainer member 119 proximate the upper end 104 of the surgical instrument holder 100, the retainer member 119 being configured to retain the surgical instrument 5 at a given insertion depth within the surgical instrument receptacle 120. More specifically, the retainer member 119 can limit a passage of the surgical instrument 5 through the surgical instrument receptacle 120 to retain the surgical instrument 5 therein at the given insertion depth. Thus, the retainer member 119 can be shaped and sized to interfere with a portion of the surgical instrument 5 having a width that is larger than a retainer member width of the retainer member 119. For instance, in some implementations, the retainer member 119 can have a retainer member width that is smaller than the widest transverse instrument width T of the surgical instrument 5. In some implementations, the resulting interference between the retainer member 119 and the surgical instrument 5 can prevent an insertion of the graspable portion 6 of the surgical instrument 5 into the surgical instrument receptacle 120 to facilitate a grasping of the surgical instrument 5 by the surgical care practitioner as described above when defining the retainer member width being smaller than the widest transverse instrument width T of the surgical instrument 5. In the implementation shown in FIG. 1, the retainer member 119 takes the form of a C-shaped member including a stem portion and two arcuate arms extending outwardly from the stem portion to define the retainer member width therebetween. In some implementations, the stem portion of the retainer member 119 can be removably engageable with the peripheral wall 110, allowing various sizes of retainer members 119 to be selected in accordance with a given surgical instrument 5 intended to be received in the surgical instrument receptacle 120. In other implementations, the retainer member 119 can take the form of a ring removably engageable with the peripheral wall 110, to modify the effective diameter or width of the surgical instrument receptacle 120 in accordance with the selected surgical instrument 5 it is intended to receive. In alternative implementations, the retainer member 119 can be a grid having an opening width smaller than the widest transverse instrument width T of the surgical instrument 5 while enabling the remainder of the surgical instrument 5 to pass therethrough and be inserted in the surgical instrument receptacle 120.

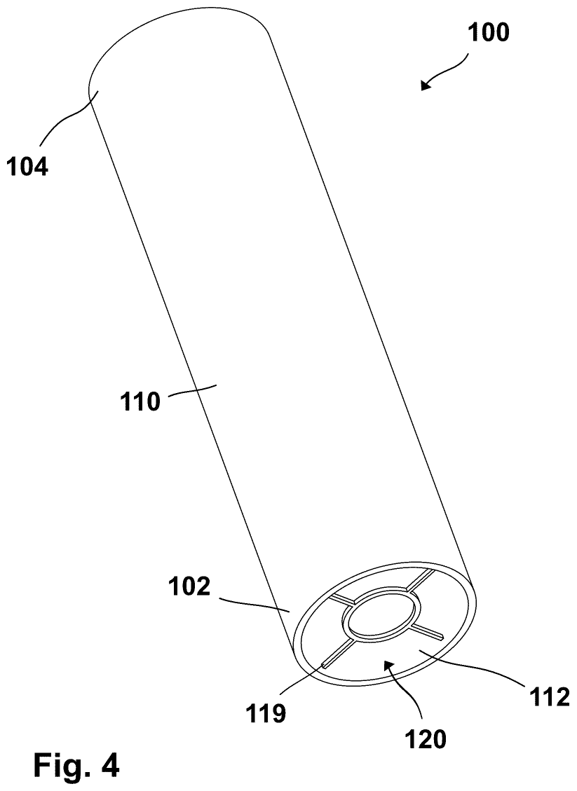

Referring now to FIG. 4, another implementation of the retainer member 119 is shown. In the illustrated implementation, the retainer member 119 is provided in a lower portion of the surgical instrument holder 100, such as a portion of the peripheral wall 110 proximate the lower end 102 of the surgical instrument holder 100. In such implementations, the retainer member 119 can be shaped and sized to interfere with a lower portion of the surgical instrument 5 having a given transverse instrument width, such as the transverse instrument width I shown in FIG. 1, that is larger than the retainer member width of the retainer member 119. The retainer member 119 can take the form of a ring removably engageable with the peripheral wall 110 near the lower end 102 of the surgical instrument receptacle 120 to modify the effective diameter, or effective width, of the surgical instrument receptacle 120 in accordance with the selected surgical instrument 5 that it is intended to receive, to limit further insertion of the surgical instrument 5 through the surgical instrument receptacle 120. In alternative implementations, the retainer member 119 can be a grid having an opening width smaller than the given transverse instrument width I of the surgical instrument 5. In some implementations, the retainer member 119 can be engaged with the peripheral wall 110 or formed with the peripheral wall 110 as a single component, i.e., integral with the peripheral wall 110.

### Attachment Features of the Surgical Assistance System

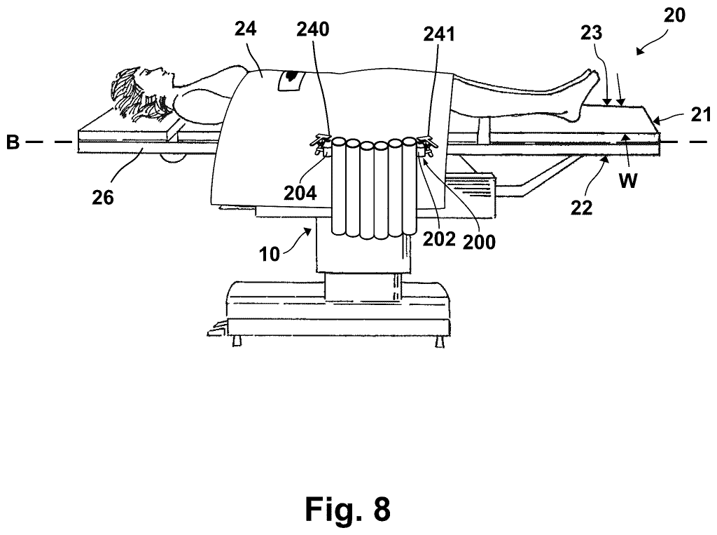

The surgical assistance system 10 further includes one or more additional features configured to enable the surgical assistance system 10 to engage a surgical table 20 (shown in FIGS. 8 to 11) or a component thereof. In some implementations, the surgical assistance system 10 can engage the surgical table 20 in a region of the surgical table 20 proximate the operative position of the surgical care practitioner so as to enable the surgical care practitioner to engage or grasp one or more of the surgical instruments 5 retained by the surgical assistance system 10 without requiring the surgical care practitioner to shift their body or extend their arms away from the surgical table.

### Surgical Assistance System Including a Brace

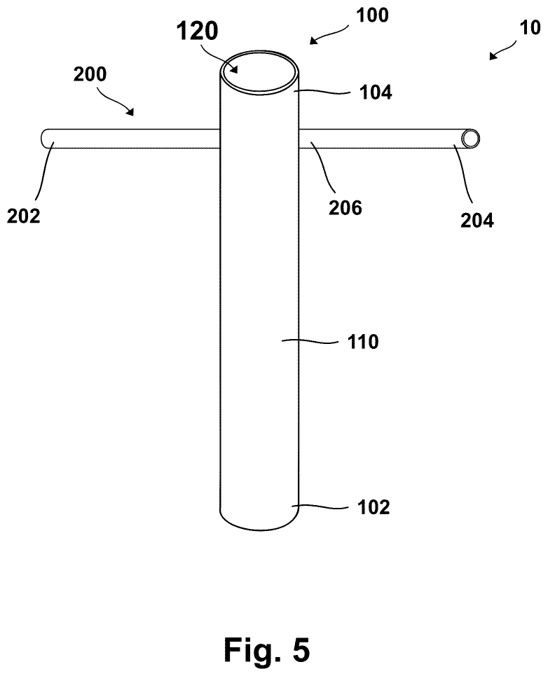

Referring to FIG. 5, an implementation of the surgical assistance system 10 is shown, the surgical assistance system 10 including a brace 200 for hanging the surgical instrument holder 100 from the surgical table 20 in an upright disposition. The brace 200 can be fitted to the surgical table 20 to provide support to the surgical instrument holder 100. In some implementations, the brace 200 can be elongated and configured to extend along at least a portion of a longitudinal side 22 of the surgical table 20 (shown for instance in FIG. 8). In the implementation shown, the brace 200 includes first and second end portions 202, 204 engageable with the surgical table 20, and a central portion 206 extending between the first and second end portions 202, 204. It is to be understood that the expression “end portion”, as used herein, is intended to refer to an arbitrary portion proximate the longitudinal extremities of the brace 200 and is not necessarily intended to refer to the furthest point of the brace 200. In some implementations, the surgical instrument holder 100 includes a brace engaging feature 130 (shown in FIG. 1) engageable with the brace 200 for securing the surgical instrument holder 100 to the brace 200 in a hanging configuration. Examples of a suitable brace engaging feature 130 include a clip, a hook, and a magnetic connector, among others.

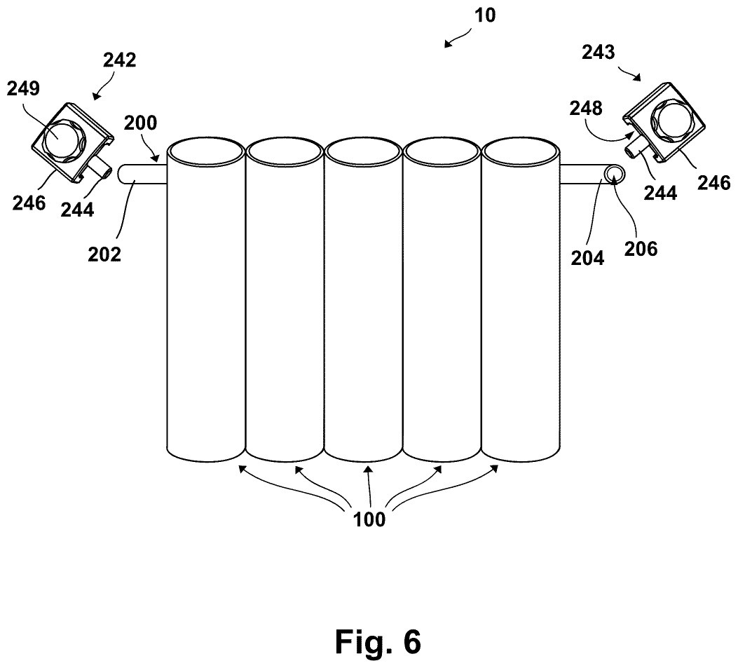

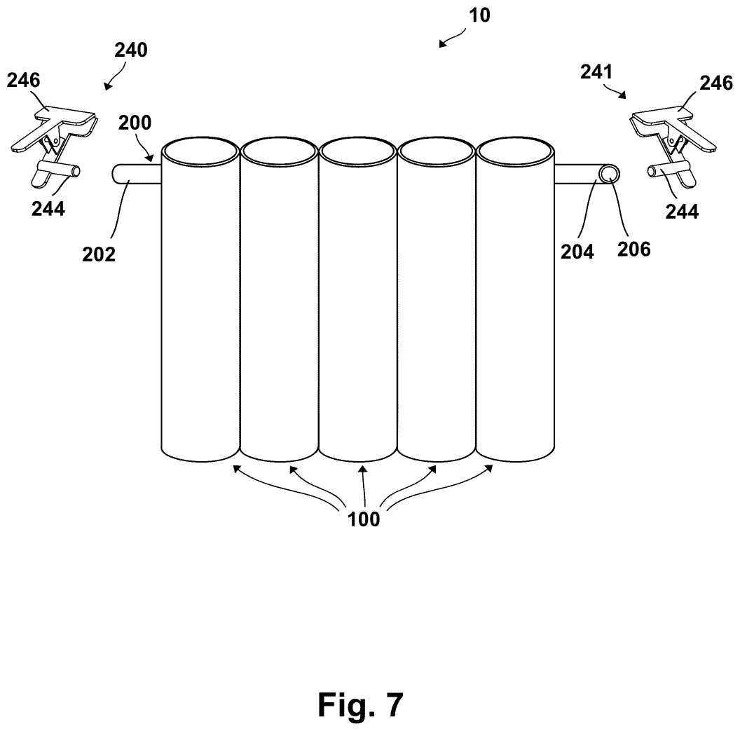

Although a single surgical instrument holder 100 is shown in the implementation of the surgical assistance system 10 shown in FIGS. 1 to 5, in other implementations, the surgical assistance system 10 can include any other suitable number of surgical instrument holders 100. In that respect, FIGS. 6 and 7 illustrate implementations of the surgical assistance system 10 that include multiple surgical instrument holders 100 provided adjacent to each other, i.e., in series and in an end-to-end fashion, to form an array of surgical instrument holders 100. In the illustrated implementations shown in FIGS. 6 and 7, the surgical assistance system 10 includes five surgical instrument holders 100, each of the surgical instrument holders 100 being configured to receive a corresponding surgical instrument 5 therein. It will be understood that, in other implementations, the surgical assistance system 10 can include any other desirable number of surgical instrument holders 100 so as to receive some or all of the surgical instruments 5 required for a particular surgical procedure.

In some implementations, the surgical assistance system 10 can be a modular assembly configured to include a variable number of surgical instrument holders 100. Accordingly, the modular surgical assistance system 10 can be modified to increase or reduce the number of surgical instrument holders 100 in accordance with the needs of a particular surgical procedure. In such implementations, each of the surgical instrument holders 100 can include a removably engageable brace engaging feature 130, exemplified in FIG. 1, configured to enable a selective attachment and detachment of each respective surgical instrument holder 100 in accordance with the needs of a particular surgical procedure.

The brace 200 can be made of any rigid, semi-rigid or flexible material suitable for supporting a weight of the one or more surgical instrument holders 100 and the surgical instruments 5 configured to be received therein. Examples of suitable materials that can be used for the brace 200 include aluminum, stainless steel, titanium and nitinol, polyether ether ketone, polyphenylsulfone, polyoxymethylene, and polypropylene. In some implementations, the brace 200 can be made of a sterilizable material to further prevent contamination during the surgical procedure.

In some implementations, the surgical assistance system 10 can be configured to engage the surgical table 20 at a location within the sterilized environment or within the unsterilized environment of the surgical table 20. For instance, in some implementations, the surgical assistance system 10 can include engaging members configured to enable engagement of the brace 200 with a sterilized surgical drape 24 of the surgical table 20 and/or an unsterilized siderail 26 of the surgical table 20. Referring to FIG. 8, an implementation of the surgical assistance system 10 is shown including first and second drape engaging members 240, 241 configured to engage the first and second end portions 202, 204 of the brace 200, respectively, with the surgical drape 24. In the illustrated implementation, each of the first and drape engaging members 240, 241 is exemplified as a clip including opposing jaws resiliently biased together for retaining a portion of the surgical drape therebetween and thus engaging the brace 200 with the surgical drape 24. In some implementations, the opposing jaws can include interlocking features, such as interlocking serrations or the like, to improve a grip of the first and drape engaging members 240, 241 when engaging the surgical drape 24.

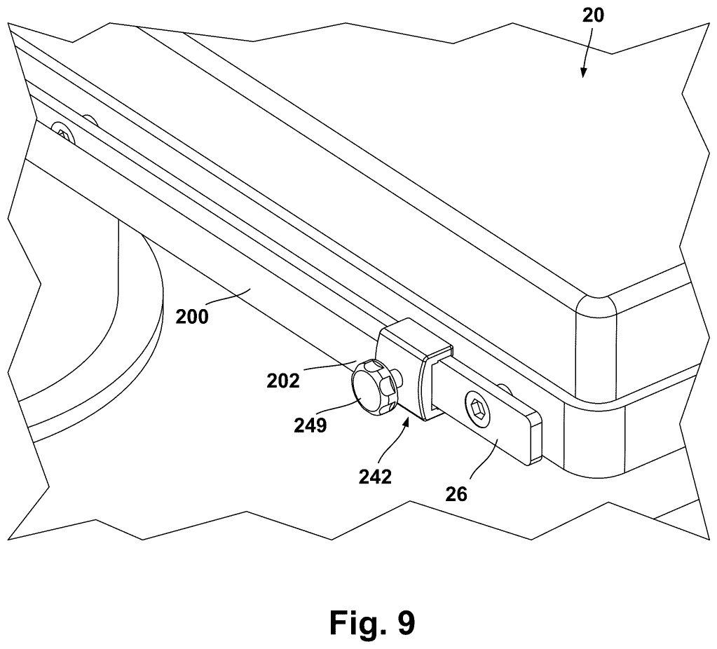

Referring to FIG. 9, an implementation of the surgical assistance system 10 is shown, with the brace 200 being configured to engage the surgical table 20 at a location within the unsterilized environment. In the implementation shown, the surgical assistance system 10 includes a brace 200 engaged with the unsterilized siderail 26 of the surgical table 20. In such implementations, the surgical assistance system 10 can include first and second siderail engaging members 242, 243 (shown in FIG. 6) configured to secure the first and second end portions 202, 204 of the brace 200, respectively, to the siderail 26 of the surgical table 20. Each of the first and second siderail engaging members 242, 243 can be a siderail clamp configured to engage the siderail 26 of the surgical table 20 at any suitable location along a length of the siderail 26. For instance, in the implementation shown in FIG. 9, the first siderail engaging member 242 is shown engaging the siderail 26 of the surgical table 20 near a distal end thereof. In some implementations, the first and second siderail engaging members 242, 243 can define a rail channel 248 (shown in FIG. 6) shaped and sized to slidingly receive the siderail 26. In such implementations, the first and second siderail engaging members 242, 243 can be positioned at any desirable location along the siderail 26 by sliding the respective first or second siderail engaging member 242, 243 along the length of the siderail 26. When the first and second siderail engaging members 242, 243 are positioned at the desired location, the first and second siderail engaging members 242, 243 can engage the siderail 26 to restrict the sliding along the siderail 26. To do so, each of the first and second siderail engaging members 242, 243 can include a hand screw 249 being operable to engage the siderail 26 of the surgical table 20. In other implementations, the first and second siderail engaging members 242, 243 can include any other suitable fasteners for engaging the siderail 26, such as screws, bolts, or the like.

Referring back to FIGS. 6 and 7, in order to secure the engaging members 240, 241, 242, 243 to the brace 200, each of the engaging members 240, 241, 242, 243 can include a first coupling end 244 configured to engage the first and second end portions 202, 204 of the brace 200, and a second coupling end 246 configured to releasably engage the surgical drape 24 or the siderail 26. In the illustrated implementation, the first coupling end 244 of the first and second clamps 240, 242 defines a male member insertable in a female cavity 206 of the brace 200 to engage the first and second end portions 202, 204 of the brace 200. It is to be understood that the shape and configuration of the engaging members 240, 241, 242, 243 can vary from the illustrated example shown in FIGS. 6 and 7. For instance, in a reverse configuration, the first coupling end 244 of the first and second clamps 240, 242 can define a female cavity into which a male member of the brace 200 can be inserted to engage the first and second end portions 202, 204 of the brace 200, among other options, among other examples.

In some implementations, the surgical assistance system 10 can engage the surgical table 20 along the longitudinal side 22 of the surgical table 20 to position the array of surgical instrument holders 100 adjacent, or spaced-apart, relative to the operative position of the surgical care practitioner along a longitudinal axis B of the surgical table 20, as will be described in greater detail below. Alternatively, in some implementations, the surgical assistance system 10 can engage the surgical table 20 to position the array of surgical instrument holders 100 at a same location along the longitudinal axis B of the surgical table 20 where the operative position of the surgical care practitioner is, with at least a portion of the array of surgical instrument holders 100 being disposed between the surgical care practitioner and the surgical table 20 during the surgical procedure.

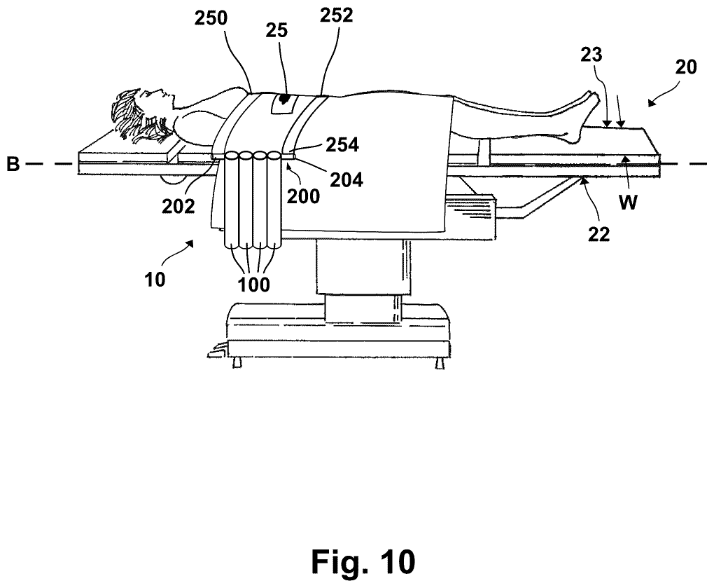

Referring to FIG. 10, a surgical assistance system 10 in accordance with another implementation is shown. The surgical assistance system 10 includes first and second support bands 250, 252. The first and second support bands 250, 252 are configured to extend across at least a portion of a width W of the surgical table 20 between first and second longitudinal sides 22, 23 of the surgical table 20 to support the brace 200, and thus also indirectly supporting the one or more surgical instrument holders 100 and the one or more surgical instruments 5 that are engaged with the brace 200. In order to do so, each of the first and second support bands 250, 252 can include a first band end 254 engageable with the first and second end portions 202, 204 of the brace 200, respectively, to position the one or more surgical instrument holders 100 along the longitudinal side 22 of the surgical table 20. In some implementations, each of the first and second support bands 250, 252 can further include a second band end (not shown) engageable with the surgical table 20 on a side of the surgical table 20 opposite the brace 200, for instance via the siderail 26, or with the surgical drape 24. In other implementations, the second band end of each of the first and second support bands 250, 252 can be engageable with an additional brace and associated array of surgical instrument holders 100, as will be described in greater detail below. It is to be understood that the expression “band end”, as used herein, is intended to refer to an arbitrary portion proximate the longitudinal extremities of the corresponding first and second support bands 250, 252 and is not necessarily intended to refer to the furthest point thereof.

In the implementation shown in FIG. 10, the first and second support bands 250, 252 are configured to extend over a portion of a body of a patient positioned on the surgical table 20, such as over a torso of the patient, with the first and second support bands 250, 252 being supported by the body of the patient. For instance and as shown in FIG. 10, the first and second support bands 250 can be placed onto the surgical drape 24 of the surgical table 20, with the brace 200 and array of surgical instrument holders 100 being positioned on the side of the surgical table 20 without further engagement therewith. In such implementations, each of the first and second support bands 250, 252 can include an elongated strip of material having a longitudinal dimension suitable for extending across the body of the patient. In some implementations, each of the first and second support bands 250, 252 can further have a transverse width suitable for distributing a weight of the surgical assistance system 10 across a sufficient portion of the body of the patient, which can contribute to preventing bruising or a restriction of blood circulation in the patient.

In some implementations and as shown in FIG. 10, the first and second support bands 250, 252 can be longitudinally spaced apart along the longitudinal axis B of the surgical table 20 once the surgical assistance system 10 is engaged with the surgical table 20. For instance, the first and second support bands 250, 252 can be positioned on opposite sides of a surgical site 25 on the patient.

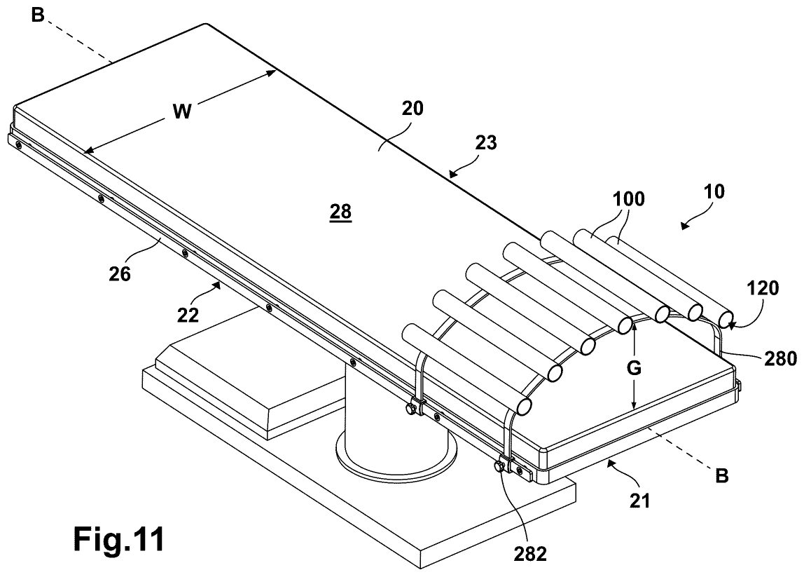

In some implementations, the surgical assistance system 10 can be configured for a surgical procedure or a medical procedure involving the surgical care practitioner being placed in an operative position located adjacent to a transverse side 21 of the surgical table 20. Examples of such procedures can include for instance gynecological procedures, among others. Referring to FIG. 11, an implementation of the surgical assistance system 10 is shown including a brace 280 configured to extend across at least a portion of the width W of the surgical table 20 between the first and second longitudinal sides 22, 23 of the surgical table 20, upwardly spaced-apart from a top surface 28 of the surgical table 20 and a patient supported by the surgical table 20, the top surface 28 of the surgical table 20 referring to the surface of the surgical table 20 configured to support a patient during a surgical procedure. In such implementations, the array of surgical instrument holders 100 can enable providing access to the one or more surgical instruments 5 by the surgical care practitioner along the transverse side 21 of the surgical table 20. In some implementations, the brace 280 can be a rigid or semi-rigid brace configured to be vertically spaced apart, i.e., upwardly spaced apart, relative to the top surface 28 of the surgical table 20 to define a brace gap G sufficiently large to enable a repositioning of the patient during the surgical or medical procedure and facilitate access of the surgical instruments 5 by the surgical care practitioner. The brace gap G can also be selected such that once the patient is positioned on the top surface 28 of the surgical table 20, the brace 280 remains spaced apart from the patient, in an upward direction. In some implementations, the brace 280 can be configured to provide support to one or more surgical instrument holders 100 placed in a substantially horizontal configuration or angled configuration with respect to the horizontal axis. Thus, in contrast to the brace 200 described above that is configured to support the surgical instrument holders 100 in a hanging configuration such that the surgical instrument holders 100 extend substantially vertically, the one or more surgical instrument holders 100 (and by association the respective surgical instrument receptacle 120 of the one or more surgical instrument holders 100) can extend substantially horizontally along the longitudinal axis B of the surgical table 20 or at a suitable angle relative to the longitudinal axis B of the surgical table 20 when engaged with the brace 280 so as to receive each corresponding surgical instrument 5 when deposited in a forward motion into the respective surgical instrument receptacle 120. In the illustrated implementation, the surgical assistance system 10 further includes siderail engaging members 282 similar to the siderail engaging members 242, 243 described above, and configured to secure the brace 280 to the surgical table 20 via the surgical rail 26.

In some implementations, the brace 280 can include a plurality of components configured to be assembled to deploy the brace 280 in its operative configuration prior to a surgical procedure, and subsequently collapsed when no longer in use. The brace 280 can thus be configured in one of a collapsed configuration and a deployed configuration, with the deployed configuration corresponding to the operative configuration. In some implementations, the components of the brace 280 can remain interconnected when the brace 280 is in the collapsed configuration with the brace 280 occupying a smaller volume. In other implementations, the components of the brace 280 can be disconnected from one another when the brace 280 is in the collapsed configuration. In some implementations, the brace 280 can be a freestanding brace when in the deployed configuration, in that it does not need to be engaged with the surgical table 20 or another structure for stability. In some implementations, the brace 280 can have a telescopic configuration enabling the brace 280 to transition from the collapsed configuration to the deployed configuration.

### Attachment Components of the Surgical Assistance System

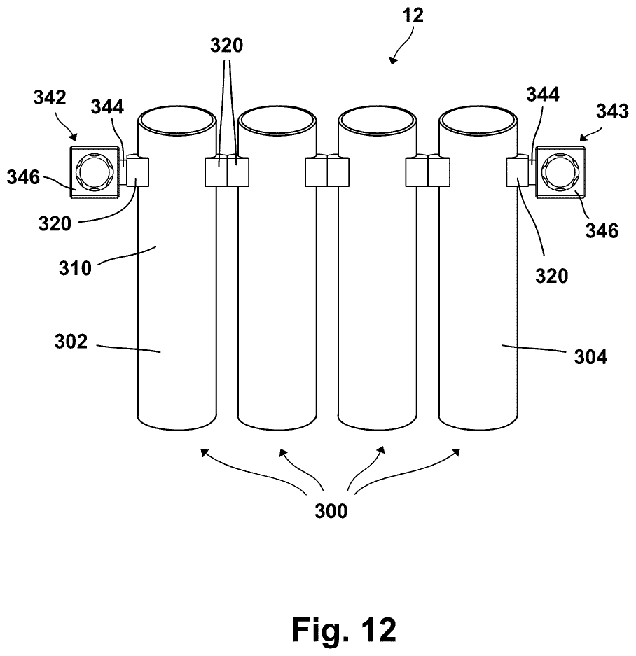

Referring to FIG. 12, an implementation of a surgical assistance system 12 is shown. In this example, the surgical assistance system 12 includes a plurality of surgical instrument holders 300 provided adjacent to each other as an array, each surgical instrument holder 300 being removably engageable with an adjacent one of the surgical instrument holders 300 to form the array of surgical instrument holders 300. Although not described in further detail in the present section, it is to be understood that the features described above with respect to the surgical assistance system 10 and the surgical instrument holders 100 are also applicable to the surgical assistance system 12 and the surgical instrument holders 300. A brief overview of the additional features of the surgical instrument holders 300 is presented below.

In the implementation shown, each of the surgical instrument holders 300 includes an attachment component 320 coupled with a peripheral wall 310 of the surgical instrument holder 300. Alternatively, the attachment component 320 can be integral with the peripheral wall 310 of the surgical instrument holder 300. The attachment component 320 of a first surgical instrument holder 300 is removably engageable with a corresponding attachment component 320 of a second surgical instrument holder 300 located adjacent to the first surgical instrument holder and so on, to form the array of surgical instrument holders 300. In such implementations, when each of the surgical instrument holders 300 is removably engageable with an adjacent surgical instrument holder 300, the resulting array of surgical instrument holders 300 can be self-supporting and a brace extending between the farthermost surgical instrument holders 300 of the array of surgical instrument holders 300 can optionally be omitted. The attachment components 320 can be any suitable attachment connector suitable for engaging with a corresponding attachment component 320 of an adjacent one of the surgical instrument holders 300 and for at least partially supporting a weight of an adjacent one of the surgical instrument holders 300 and the surgical instrument 5 configured to be received therein. Examples of suitable attachment components include a clip, a hook, and a magnetic connector, among others.

In some implementations, the surgical assistance system 12 can be configured to engage the surgical table 20 at a location within the sterilized environment or within the unsterilized environment of the surgical table 20, similarly to the surgical assistance system 10 described above. More specifically, in some implementations, a foremost surgical instrument holder 302 and an endmost surgical instrument holder 304 of the array of surgical instrument holders 300 can be removably engageable with one of the sterilized surgical drape 24 of the surgical table 20 and/or the unsterilized siderail 26 of the surgical table 20. In the implementation shown in FIG. 12, the surgical assistance system 12 includes first and second siderail engaging members 342, 343 configured to removably secure the foremost and endmost surgical instrument holders 302, 304, respectively, to the siderail 26 of the surgical table 20, similarly to the first and second siderail engaging members 242, 243 described above. Each of the first and second siderail engaging members 342, 343 can include a first coupling end 344 configured to engage the corresponding attachment component 320. More specifically, in the illustrated implementation, the first coupling end 344 of the first and second siderail engaging members 342, 343 is configured to engage the attachment component 320 of the foremost and endmost surgical instrument holders 302, 304, respectively. The first and second siderail engaging members 342, 343 can further include a second coupling end 346 configured as a siderail clamp to releasably engage the siderail 26 of the surgical table 20. In other implementations, the surgical assistance system 12 can include drape engaging members configured to removably secure the foremost and endmost surgical instrument holders 302, 304, respectively, to the surgical drape 24 of the surgical table 20, similarly to the first and second drape engaging members 240, 241 described above.

### Additional Features of the Surgical Assistance System

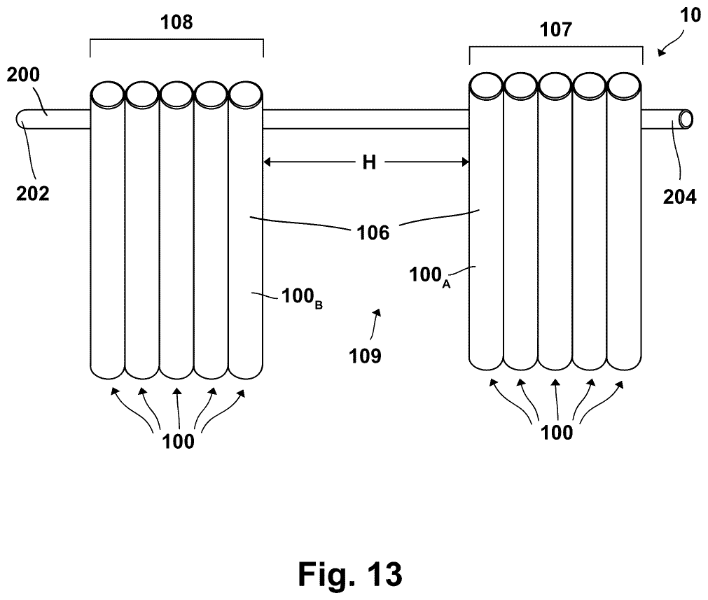

Referring to FIG. 13, another implementation of the surgical assistance system 10 is shown. In this implementation, the surgical assistance system 10 includes a pair 106 of surgical instrument holders 100 that are horizontally spaced apart from each other to define a surgical table access 109 therebetween. The surgical table access 109 can contribute to facilitate access to the surgical table 20 once the surgical assistance system 10 is engaged with the surgical table 20. In such implementations, the surgical table access 109 can have a width referred to as a horizontal spacing H measured between the two spaced-apart surgical instrument holders 100 of the pair 106 of surgical instrument holders 100, the horizontal spacing H being sufficient to enable a surgical care practitioner to position themselves between the two spaced-apart surgical instrument holders 100 of the pair 106 of surgical instrument holders 100 during a surgical procedure.

In some implementations, the pair 106 of surgical instrument holders 100 can be part of an array of surgical instrument holders 100. In some implementations, the array of surgical instrument holders 100 can include additional surgical instrument holders 100. In such implementations, the array of surgical instrument holders 100 can be said to include two subsets 107, 108 of surgical instrument holders 100, the two subsets 107, 108 being spaced apart from each other by the horizontal spacing H. For instance, in the implementation shown in FIG. 13, the array of surgical instrument holders 100 includes a first subset 107 of surgical instrument holders 100 including a first surgical instrument holder 100A of the pair 106 of surgical instrument holders 100 positioned on a righthand side of the surgical table access 109, such that when a surgical care practitioner is positioned within the surgical table access 109, the surgical care practitioner can have access to a surgical instrument received in the surgical instrument receptacle 120 of the first surgical instrument holder 100A with a right hand. In addition, in the implementation shown, the array of surgical instrument holders 100 further includes a second subset 108 of surgical instrument holders 100 including a second surgical instrument holder 100B of the pair 106 of surgical instrument holders 100 positioned on a lefthand side of the surgical table access 109, such that when the surgical care practitioner is positioned within the surgical table access 109, the surgical care practitioner can have access to a surgical instrument received in the surgical instrument receptacle of the first surgical instrument holder 100B with a left hand. It will be appreciated that, while each of the first and second subsets 107, 108 of surgical instrument holders 100 shown in the implementation illustrated in FIG. 13 includes five surgical instrument holders 100, in other implementations, the first and second subsets 107, 108 of surgical instrument holders 100 can include any other desirable number of surgical instrument holders 100. As mentioned above, the first and second surgical instrument holders 100A, 100B of the pair 106 of surgical instrument holders 100 can be horizontally spaced apart by a horizontal spacing H sufficiently large to accommodate a hip breadth of the surgical care practitioner. For instance, in some implementations, the horizontal spacing H can be at least about 45 cm, or at least about 60 cm.

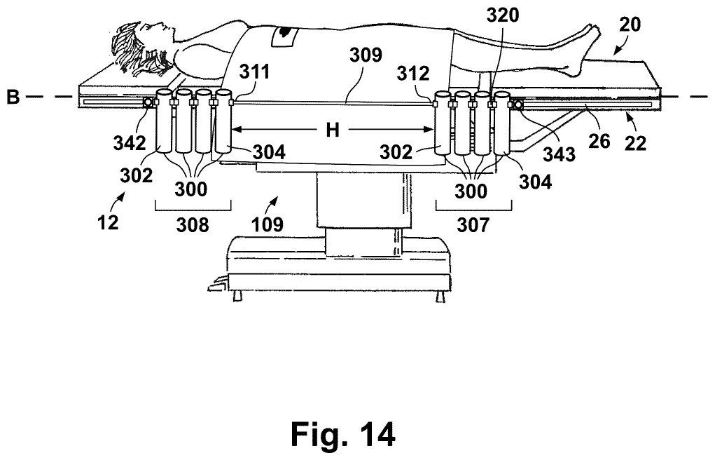

In the implementation illustrated in FIG. 13, the surgical assistance system 10 includes a brace 200 to support the array of surgical instrument holders 100 provided as the two subsets 107, 108 of surgical instrument holders 100 spaced apart from each other by the horizontal spacing H. In the implementation shown in FIG. 14, the surgical assistance system 12 includes two arrays of surgical instrument holders 300 engaged with the surgical table 20 so as to be horizontally spaced apart from each other to define the surgical table access 109 therebetween. In such implementations, each of the arrays of surgical instrument holders 300 can be said to form a subset 307, 308 of surgical instrument holders 300, the two subsets 307, 308 being spaced apart from each other by the horizontal spacing H. In the implementation shown in FIG. 14, the surgical assistance system 12 includes first and second siderail engaging members 342, 343 for securing each of the subsets 307, 308 to the surgical table 20 via the siderail 26. It will be understood that, in other implementations, the surgical assistance system 12 can include drape engaging members similar to the drape engaging members 240, 241 described above for securing each of the subsets 307, 308 to the surgical table 20 via the surgical drape 24.

As described above, each of the surgical instrument holders 300 can include attachment components 320 to engage adjacent surgical instrument holders 300 to form each of the subsets 307, 308 of surgical instrument holders 300. In such implementations, the resulting subsets 307, 308 can be said to be self-supporting, with each of the surgical instrument holders 300 being supported by an adjacent surgical instrument holder 300 or one of the first and second siderail engaging members 342, 343. When the arrays of surgical instrument holders 300 are self-supporting, the brace 200 can optionally be omitted. Each of the two subsets 107, 108 of surgical instrument holders 300 can thus engage the surgical table 20 at respective positions on opposite sides of the operative position to define the horizontal spacing H therebetween. In some implementations, the surgical assistance system 12 can further include a spacing member 309 (shown in FIG. 14) configured to engage the foremost and endmost surgical instrument holders 302, 304 of the subsets 307, 308 of surgical instrument holders 300, respectively, to space apart the two subsets 307, 308 of surgical instrument holders 300 at a predefined distance corresponding to the horizontal spacing H. In some implementations, the spacing member 309 can include an elongated rigid or semi-rigid member having opposite ends 311, 312. Each of the opposite ends 311, 312 of the spacing member 309 can engage the foremost and endmost surgical instrument holders 302, 304 of the subsets 307, 308 of surgical instrument holders 300, respectively, via for instance their respective attachment components 320.

In some implementations, the surgical assistance system 10, 12 can include surgical instrument holders 100, 300 configured to be positioned on opposing sides of the surgical table 20. For instance, in implementations including the first and second support bands 250, 252 such as shown in FIG. 10, each of the first and second support bands 250, 252 can include opposite band ends engageable with at least one surgical instrument holder 100, 300. In such implementations, the surgical instruments holders 100, 300 and their content can be configured to have similar weights so as to prevent a displacement of the surgical assistance system 10, 12 towards one of the first and second longitudinal sides 22, 23 of the surgical table 20. In implementations including multiple surgical instrument holders 100, 300 provided adjacent to each other to form an array of surgical instrument holders 100, 300, the band ends of the first and second support bands 250, 252 can be engageable with the first and second end portions 202, 204 of the brace 200 of the surgical assistance system 10, or a foremost and endmost surgical instrument holders 302, 304 of the array of surgical instrument holders 300. In such implementations, the arrays of surgical instrument holders 100, 300 configured for placement along the opposing first and second longitudinal sides 22, 23 of the surgical table 20 can be referred to as first and second arrays of surgical instrument holders 100, 300. In such implementations, each of the first and second arrays of surgical instrument holders 100, 300 can be configured similar to either one of the arrays of surgical instrument holders 100, 300 defined above. In such implementations, the first and second arrays of surgical instruments holders 100, 300 and their content can be configured to have similar weights so as to prevent a displacement of the surgical assistance system 10, 12 towards one of the first and second longitudinal sides 22, 23 of the surgical table 20.

In some implementations, when the surgical instrument holders 100 are engaged with the brace 200 in the hanging configuration and the surgical assistance system 10 is positioned along a side of the surgical table 20, the one or more surgical instrument holders 100 (and by association the respective surgical instrument receptacle 120 of the one or more surgical instrument holders 100) can extend downwardly so as to receive the surgical instrument when deposited in a downward motion into the surgical instrument receptacle 120. In some implementations, the surgical instrument holder 100 can extend downwardly at a slight angle with respect to a vertical axis once the surgical assistance system 10 is engaged with the surgical table 20. For instance, the surgical instrument holder 100 can extend downwardly and away from the operative position of the surgical care practitioner at a tilt angle a with respect to the vertical axis to facilitate an insertion of the surgical instrument 5 into the surgical instrument receptacle 120. In some implementations, the surgical instrument holder 100 can extend downwardly at a tilt angle a of at least about 5° with respect to the vertical axis once the surgical assistance system 10 is positioned along a side of the surgical table 20. In other implementations, the surgical instrument holder 100 can extend downwardly at a tilt angle a of at least about 10° with respect to the vertical axis once the surgical assistance system 10 is positioned along a side of the surgical table 20. In still other implementations, the surgical instrument holder 100 can extend downwardly at a tilt angle a of at least about 20° with respect to the vertical axis once the surgical assistance system 10 is positioned along a side of the surgical table 20.

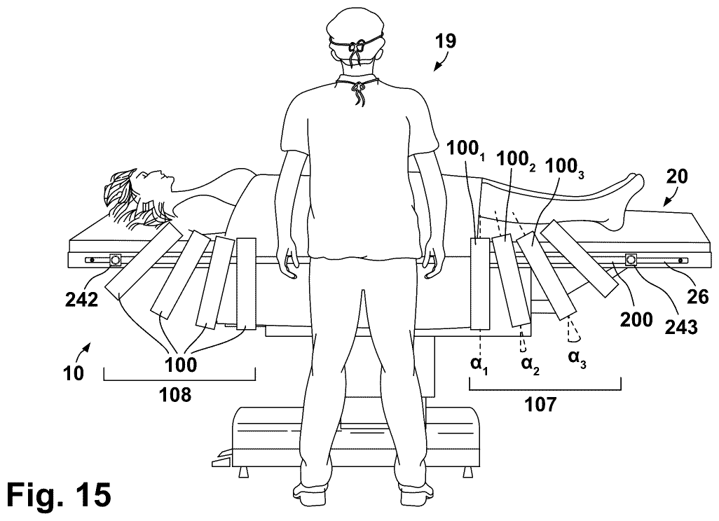

In some implementations, when the surgical assistance system 10 includes multiple surgical instrument holders 100, the surgical instrument holders 100 can extend downwardly from the surgical table 20 in a substantially parallel direction relative to each other once the surgical assistance system 10 is positioned along a side of the surgical table 20. Referring to FIG. 15, in some implementations, it may be desirable for a surgical instrument holder 100 positioned further from an operative position 19 to be angled relative to a surgical instrument holder 100 positioned closer to the operative position 19. The angle of the surgical instrument holder 100 positioned further from the operative position 19 can enable the medical practitioner to insert a surgical instrument 5 into the corresponding surgical instrument receptacle 120 by extending their arm without requiring the surgical care practitioner to significantly shift their body away from the operative position 19. More specifically, it may be desirable to substantially align the longitudinal axis of one or more of the surgical instrument holders 100 with a long axis of the surgical care practitioner's arm thereby enabling the surgical care practitioner to deposit or retrieve a surgical instrument 5 via an extension or flexion of the arm without shifting their body away from the operative position 19. Accordingly, in some implementations, adjacent ones of the surgical instrument holders 100 may extend downwardly at differing angles. In the implementation shown in FIG. 15, the surgical assistance system 10 is shown including surgical instrument holders 100 spaced apart from each other to define first and second subsets 107, 108 of surgical instrument holders 100. A firstmost surgical instrument holder 1001 of the first subset 107 of surgical instrument holders 100 being closest to the operative position 19 on a righthand side of the surgical care practitioner extends downwardly from the surgical table 20 in a substantially vertical direction (i.e., extending downwardly at a tilt angle α1 of about 0° relative to the vertical axis). A second surgical instrument holder 1002 of the first subset 107 of surgical instrument holders 100 adjacent to the firstmost surgical instrument holder 1001 extends downwardly and away from the operative position 19 at a tilt angle α2 greater than the tilt angle α1 of the firstmost surgical instrument holder 1001 so as to facilitate an insertion of a surgical instrument into the second surgical instrument holder 1002. Similarly, a third surgical instrument holder 1003 of the first subset 107 of surgical instrument holders 100 adjacent to the second surgical instrument holder 1002 extends downwardly and away from the operative position 19 at a tilt angle α3 greater than the tilt angle α2 of the second surgical instrument holder 1002. In this manner, each consecutive surgical instrument holder 100 of the first and second subsets 107, 108 of surgical instrument holders 100 can extend downwardly and away from the operative position 19 at progressively increasing tilt angles relative to the vertical axis in accordance with a distance from the operative position 19 to facilitate insertion of the surgical instrument 5 into the corresponding surgical instrument receptacle 120.

In some implementations, each of the surgical instrument holders 100, 300 can include modular parts configured to be assembled to form the respective surgical instrument holder 100, 300. For instance, in some implementations, the peripheral wall 110 of the surgical instrument holders 100, 300 can be formed of at least two portions, each portion corresponding to a modular part. The two portions of the peripheral wall 110 are configured to be assembled to each other to form a resulting surgical instrument holder 100, 300. With such a configuration of the surgical instrument holders 100, 300, a given modular part of the surgical instrument holders 100, 300 can be removed from the surgical instrument holders 100, 300 to be separately cleaned, for instance in a medical sterilizer, such as an autoclave. In addition, the option of being able to remove a given modular part of the surgical instrument holders 100, 300 can further facilitate a repair of the given modular part of the surgical instrument holders 100, 300 in the event of damage or excessive wear.

To provide a more concise description, some of the quantitative expressions provided herein are qualified with the term “about”. It will be understood that whether the term “about” is used explicitly or not, every quantity recited herein is meant to refer to an actual given value, and it is also meant to refer to the approximation to such given value that would reasonably be inferred by a person of ordinary skill in the art, including approximations due to the experimental and/or measurement conditions for such given value.

Several alternative embodiments and examples have been described and illustrated herein. The embodiments of the invention described above are intended to be exemplary only. A person of ordinary skill in the art would appreciate the features of the individual embodiments, and the possible combinations and variations of the components. A person of ordinary skill in the art would further appreciate that any of the embodiments could be provided in any combination with the other embodiments disclosed herein. It is understood that the invention may be embodied in other specific forms without departing from the central characteristics thereof. The present examples and embodiments, therefore, are to be considered in all respects as illustrative and not restrictive, and the invention is not to be limited to the details given herein. Accordingly, while the specific embodiments have been illustrated and described, numerous modifications come to mind. The scope of the invention is therefore intended to be limited solely by the scope of the appended claims.

1. A surgical assistance system for use in cooperation with a surgical table, the surgical assistance system comprising:
surgical instrument holders provided adjacent to each other to form an array configured for placement along a first longitudinal side of the surgical table, each surgical instrument holder comprising:
a peripheral wall having an upper end surface, the peripheral wall defining a surgical instrument receptacle having a surgical instrument receiving opening and being configured to extend downwardly once the surgical assistance system is engaged with the surgical table, the surgical instrument receptacle being configured to receive at least a portion of a surgical instrument therein.

2. The surgical assistance system of claim 1, further comprising a brace for hanging the array of surgical instrument holders, the brace being configured to extend along at least a portion of the first longitudinal side of the surgical table and including first and second end portions engageable with the surgical table, each one of the surgical instrument holders including a brace engaging feature engageable with the brace.

3. The surgical assistance system of claim 2, further comprising first and second siderail engaging members configured to secure the first and second end portions of the brace, respectively, to a siderail of the surgical table.

4. The surgical assistance system of claim 2, further comprising first and second drape engaging members configured to secure the first and second end portions of the brace, respectively, to a surgical drape of the surgical table.

5. The surgical assistance system of claim 1, wherein one of the surgical instrument holders is removably engageable with an adjacent one of the surgical instrument holders of the array of surgical instrument holders.

6. The surgical assistance system of claim 5, wherein the one of the surgical instrument holders comprises an attachment component coupled to the peripheral wall, the attachment component enabling the removable engagement of the one of the surgical instrument holders with the adjacent one of the surgical instrument holders.

7. The surgical assistance system of claim 5, wherein a foremost one of the surgical instrument holders and an endmost one of the surgical instrument holders of the array of surgical instrument holders are removably engageable with the surgical table.

8. The surgical assistance system of claim 7, further comprising first and second siderail engaging members configured to removably secure the foremost one of the surgical instrument holders of the array of surgical instrument holders and the endmost one of the surgical instrument holders of the array of surgical instrument holders, respectively, to a siderail of the surgical table.

9. The surgical assistance system of claim 7, further comprising first and second drape engaging members configured to removably secure the foremost one of the surgical instrument holders of the array of surgical instrument holders and the endmost one of the surgical instrument holders of the array of surgical instrument holders, respectively, to a surgical drape of the surgical table.

10. The surgical assistance system of claim 1, wherein the array of surgical instrument holders is a first array of surgical instrument holders, the surgical assistance system further comprising a second array of surgical instrument holders configured for placement along a second longitudinal side of the surgical table opposite the first longitudinal side of the surgical table.

11. The surgical assistance system of claim 10, further comprising first and second support bands configured to extend across at least a portion of a width of the surgical table between the first and second longitudinal sides of the surgical table, the first and second support bands being longitudinally spaced apart along a longitudinal axis of the surgical table once the surgical assistance system is engaged with the surgical table, wherein each of the first and second arrays of surgical instrument holders is engageable with the first and second support bands.

12. The surgical assistance system of claim 1, wherein the surgical instrument receiving opening of a respective one of the surgical instrument holders has an opening width larger than a transverse instrument width of the surgical instrument to receive the surgical instrument free of interference within the surgical instrument receptacle of the respective one of the surgical instrument holders.

13. The surgical assistance system of claim 12, wherein the opening width of the surgical instrument receiving opening is smaller than a widest transverse instrument width of at least a portion of the surgical instrument to enable the at least a portion of the surgical instrument to abut the upper end surface of the respective one of the surgical instrument holders while enabling insertion of a remainder of the surgical instrument into the surgical instrument receptacle of the respective one of the surgical instrument holders.

14. The surgical assistance system of claim 1, wherein at least one of the surgical instrument holders is made of an electrically non-conductive material.

15. The surgical assistance system of claim 1, wherein the peripheral wall of at least one of the surgical instrument holders includes modular parts configured to be assembled to form a corresponding one of the surgical instrument holders.

16. The surgical assistance system of claim 1, wherein a pair of surgical instrument holders of the array of surgical instrument holders are horizontally spaced apart and together define a horizontal spacing providing access to the surgical table once the surgical assistance system is engaged with the surgical table.

17. The surgical assistance system of claim 1, wherein adjacent surgical instrument holders of the array of surgical instrument holders extend downwardly at progressively increasing angles with respect to a vertical axis once the surgical assistance system is engaged with the surgical table to facilitate an insertion of the surgical instrument into the surgical instrument receptacle of each of the adjacent surgical instrument holders.

18. A surgical assistance system for use in cooperation with a surgical table having a top surface for receiving a patient thereon, the surgical assistance system comprising:
a surgical instrument holder comprising a peripheral wall defining a surgical instrument receptacle having a surgical instrument receiving opening, the surgical instrument receptacle being configured to receive at least a portion of a surgical instrument therein; and
a brace configured to extend across at least a portion of a width of the surgical table between longitudinal sides of the surgical table and to be vertically spaced-apart from the top surface of the surgical table to define a brace gap therebetween, the brace being engageable with the surgical instrument holder.

19. The surgical assistance system of claim 18, wherein the brace gap is selected such that once the patient is positioned on the top surface of the surgical table, the brace remains spaced apart from the patient, in an upward direction.

20. The surgical assistance system of claim 18, wherein the surgical instrument receptacle of the surgical instrument holder extends substantially parallelly relative to the top surface of the surgical table when the brace extends across the at least a portion of the width of the surgical table.

21. The surgical assistance system of claim 18, wherein the surgical instrument receptacle of the surgical instrument holder extends at an angle relative to the top surface of the surgical table when the brace extends across the at least a portion of the width of the surgical table.

22. The surgical assistance system of claim 18, wherein the brace is configurable between a collapsed configuration and a deployed configuration, the brace being engageable with the surgical instrument holder when in the deployed configuration.

23. The surgical assistance system of claim 22, wherein, when the brace is in the deployed configuration, the brace is a freestanding brace.

24. The surgical assistance system of claim 18, further comprising first and second siderail engaging members configured to secure first and second end portions of the brace, respectively, to a siderail of the surgical table.

25. The surgical assistance system of claim 18, further comprising first and second drape engaging members configured to secure first and second end portions of the brace, respectively, to a surgical drape of the surgical table.
